(19) United States
(12) Patent Application Publication
(10) Pub. No.: US 2024/0123021 A1
(43) Pub. Date: Apr. 18, 2024

(54) PHARMACEUTICAL COMPOSITIONS

(71) Applicant: EXTROVIS AG, Baar (CH)

(72) Inventors: Krishna Mohan CHILAKALA, Hyderabad (IN); Nagamalleswara Rao BEERAKA, Hyderabad (IN); Ramesh MANTRI, Sangareddy (IN); Hanumantha Rao KAMMA, Zurich (CH); Janos VACZI, Schwyz (CH)

(21) Appl. No.: 18/005,401

(22) PCT Filed: Jun. 25, 2022
(86) PCT No.: PCT/IB2022/055910

§ 371 Date: Jan. 13, 2023

(30) Foreign Application Priority Data
Jun. 25, 2021 (IN) 202121028519

(51) Int. Cl. A61K 38/08; A61K 9/00; A61K 9/08; A61K 47/10; A61K 47/12; A61K 47/18; A61K 47/26

(52) U.S. Cl. CPC A61K 38/08; A61K 9/0019; A61K 9/08; A61K 47/10; A61K 47/12; A61K 47/183; A61K 47/26

(57) ABSTRACT

The present invention relates to a stable parenteral composition comprising Cetrorelix or a pharmaceutically acceptable salt thereof, which is ready-to-use and does not require reconstitution prior to administration.

### FIELD OF THE INVENTION

Cetrorelix acetate was approved by USFDA in the year 2000 as the brand name Cetrotide®. Cetrotide® is presented as lyophilized powder containing Cetrorelix acetate, equivalent respectively to 0.25 and 3.0 mg Cetrorelix base. The main excipient consists of mannitol. Acetic acid and water for injection are used during the manufacturing process. Prior to subcutaneous injection, the powders are reconstituted with 1 ml or 3 ml of water for injection, which are provided in prefilled syringes.

### BACKGROUND

Cetrorelix acetate is a synthetic decapeptide having terminal acid amide group. Cetrorelix has gonadotropin releasing hormone (GnRH) antagonistic activity. It competes with natural GnRH for binding to membrane receptors on pituitary cells and thus controls the release of luteinizing hormone (LH) and follicle-stimulating hormone (FSH). It is indicated for the inhibition of premature LH surges in women undergoing controlled ovarian stimulation. Cetrorelix acetate is an analog of native GnRH with substitutions of amino acids at positions 1, 2, 3, 6, and 10.

The molecular formula is Acetyl-D-3-(2′-naphtyl)-alanine-D-4-chlorophenylalanine-D-3-(3′-pyridyl)alanine-L-serine-L-tyrosine-D-citruline-L-leucine-L-arginine-L-proline-D-alanine-amide, and the molecular weight is 1431.06, calculated as the anhydrous free base. The structural formula of Cetrorelix is as follows:

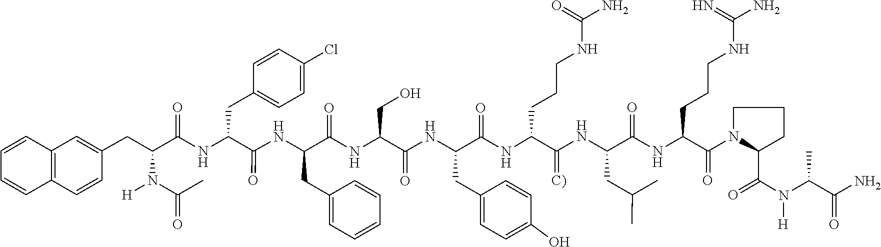

According to Cetrotide® EPAR scientific discussion, Cetrorelix acetate is thermally labile and unstable in aqueous solution. Cetrorelix in solution also tends to aggregate and gel. This is the primary reason that Cetrotide® is formulated as a lyophilized composition, that is reconstituted just prior to administration.

EP0611572B1 describes lyophilized formulation of Cetrorelix as the inventors found out that the aqueous solutions of the decapeptide are unstable. It does not disclose or teach ready-to-use (RTU) compositions of Cetrorelix.

U.S. Pat. No. 7,393,834B2 is directed to the use of lyophilized Cetrorelix for the treatment of male and female fertility disorders. It does not throw light on RTU liquid injection of Cetrorelix.

Indian patent application No. IN201821041976 discloses an aqueous injectable formulation of Cetrorelix with stabilizing agents like lactic acid, propionic acid, aspartic acid and mixtures thereof. The pH of the solution is adjusted to pH 2.5 to pH 4.5. It also discloses use of sucrose and sulfobutyl ether of beta-cyclodextrin sodium as tonicity adjusting agents.

US 20130303464A1 discloses ready-to-use Cetrorelix injection which is devoid of surfactant and uses acetic acid, having a preferred pH of 2.8 to 3.5 The applicants report that gel layer formation was not observed during filtration and aggregates were not observed in the formulation, or during stability testing.

U.S. Pat. No. 7,214,662 suggests an aqueous injectable solution of Cetrorelix using gluconic acid or its derivatives and surfactants, and reports a low tendency for formation of aggregates.

RU 2002134463 claims a dosage form for parenteral administration of peptides like Cetrorelix or their salt forms with acids such as acetic acid, gluconic acid, glucuronic acid, lactic acid, citric acid, ascorbic acid, benzoic acid or phosphoric acid, in a soluble or dispersed form. It does not expressly teach stable RTU solutions of Cetrorelix.

WO2020/254952A1 relates to stable ready to use formulation of Cetrorelix comprising glacial acetic acid, cyclodextrin and surfactant.

US20210121517A1 claims ready to use sterile parenteral dosage form comprising Cetrorelix, lactic acid, an osmotic agent and water for injection. It states that injection can be provided as a prefilled syringe or autoinjector. The application claims stable aqueous solutions of Cetrorelix comprising Cetrorelix (base) and organic acid in a weight ratio ranging from 0.47:1 to 19.23:1. The formulation is stable at a pH range of 3-5. Applicants have identified different impurities in the injectable solution of Cetrorelix. However, it is known that the use of lactic acid causes irritation and burning sensation at the site of injection, leading to patient discomfort.

### DESCRIPTION

In order to overcome aggregation and enable longer shelf-life, the first approved product Cetrotide® is supplied as lyophilized powder for reconstitution. But the lyophilized products also have some disadvantages which are as follows:
Increase in product handling and processing time
Need of sterile diluent for reconstitution
The reconstitution of lyophilized formulations is inconvenient as the reconstituted product is stable for short duration
Requirement of costly and complex instrumentation
The final formulation is expensive.

For use in long term treatments, such as those in fertility treatments, self-injectable dosage forms contribute to persistence and adherence to treatment. Lyophilized products are troublesome for self-administration, and almost always require trained healthcare professionals for reconstitution and administration. Ready-to-use (RTU) liquid solution injectable is always a better option for self-administration in such cases. Also, there are other advantages of liquid injections in terms of manufacturing like reduced processing handling time, manufacturing process is simple, and manufacturing cost is comparatively less, as the equipment and machinery are cheaper.

Cetrorelix solutions formulated with different acids and stabilizers may be stable but the use of acids in higher concentrations, and use of some acids like lactic acid, presents acceptability limitations. Hence, there is a need to develop alternate formulations of Cetrorelix. The present invention addresses this need.

The present invention relates to ready-to-use stabile aqueous compositions of Cetrorelix or its pharmaceutically acceptable salt for parenteral administration, which do not require reconstitution prior to administration.

One aspect of the invention is to provide a stable ready-to-use liquid pharmaceutical composition comprising Cetrorelix or its pharmaceutically acceptable salt, amino acid as stabilizer and other pharmaceutically acceptable excipients.

Another aspect of the present invention is to provide a stable ready-to-use liquid composition comprising Cetrorelix or its pharmaceutically acceptable salt, amino acid as stabilizer, isotonicity agent, optionally co-solvent(s) and other pharmaceutically acceptable excipients.

In some embodiments, the present invention further comprises one or more cosolvents. A “cosolvent” is a solvent which is added to the liquid pharmaceutical composition in a weight amount which is less than that of water and assists in the solubilization of Cetrorelix and/or a pharmaceutically acceptable salt thereof, thereby enhancing stability of the ready-to-use composition of the invention. Cosolvents suitable for use in the liquid pharmaceutical composition of the present invention include, but are not limited to, propylene glycol, ethanol, glycerine, polyethylene glycol and mixtures thereof.

Yet another aspect of the present invention is to provide a stable ready-to-use liquid composition comprising Cetrorelix or its pharmaceutically acceptable salt, one or more amino acids as stabilizer, acetic acid as an acidifying agent, isotonicity agent and other pharmaceutically acceptable excipients, wherein the weight ratio of Cetrorelix (base) and acetic acid is 0.08:1.

Another aspect of the present invention is to provide a ready-to-use liquid composition comprising Cetrorelix or its pharmaceutically acceptable salt, one or more amino acids as stabilizer, acetic acid as an acidifying agent, such that the composition has a pH of about 3 to about 5.5 and has no more than 3% w/w (by weight of Cetrorelix base) of total impurities at the end of its shelf-life, when stored at 2-8° C.

In another aspect of the present invention is provided a stable ready-to-use liquid composition comprising Cetrorelix or its pharmaceutically acceptable salt, one or more amino acids as stabilizer, tartaric acid as an acidifying agent, isotonicity agent and other pharmaceutically acceptable excipients, wherein the weight ratio of Cetrorelix (base) and tartaric acid is 0.25:1.

Another aspect of the present invention is to provide a ready-to-use liquid composition comprising Cetrorelix or its pharmaceutically acceptable salt, one or more amino acids as stabilizer, tartaric acid as an acidifying agent, such that the composition has a pH of about 3 to about 5.5 and has no more than 3% w/w (by weight of Cetrorelix base) of total impurities at the end of its shelf-life, when stored at 2-8° C.

In one aspect, the present invention provides a stable ready-to-use pharmaceutical composition for parenteral administration comprising
i) Cetrorelix base or a pharmaceutically acceptable salt thereof,
ii) a stabilizing agent,
iii) an acidifying agent in an amount sufficient to adjust the pH of the solution in the range of about 3 to about 5.5
iv) an isotonicity agent and
v) optionally cosolvent
   wherein after 6 months of storage at 25° C. and 60% relative humidity, the solution contains not more than 1% w/w of L-Ala-Cetrorelix impurity by weight of Cetrorelix base.

In another aspect of the present invention a stable ready-to-use pharmaceutical composition for parenteral administration is provided, comprising
i) from about 0.1 mg to about 0.5 mg/ml Cetrorelix base or a pharmaceutically acceptable salt thereof
ii) glycine as a stabilizing agent,
iii) an acidifying agent in an amount sufficient to adjust the pH of the solution in the range of about 3 to about 5.5
iv) an isotonicity agent and
v) optionally cosolvent
   wherein after 6 months of storage at 25° C. and 60% relative humidity, the solution contains not more than 1% w/w of L-Ala-Cetrorelix impurity by weight of Cetrorelix base.

In one embodiment, the present invention provides a stable ready-to-use pharmaceutical composition for parenteral administration comprising
i) Cetrorelix base or a pharmaceutically acceptable salt thereof,
ii) a stabilizing agent,
iii) an acidifying agent in an amount sufficient to adjust the pH of the solution in the range of about 3 to about 5.5
iv) an isotonicity agent and
v) optionally cosolvent
   wherein after 6 months of storage at 25° C. and 60% relative humidity, the solution contains not more than 3% w/w of total impurity by weight of Cetrorelix base.

In yet another aspect of the present invention a stable ready-to-use pharmaceutical composition for parenteral administration is provided, comprising
i) from about 0.1 mg to about 0.5 mg/ml Cetrorelix base or a pharmaceutically acceptable salt thereof
ii) glycine as a stabilizing agent,
iii) an acidifying agent in an amount sufficient to adjust the pH of the solution in the range of about 3 to about 5.5
iv) an isotonicity agent and
v) optionally cosolvent
   wherein after 6 months of storage at 25° C. and 60% relative humidity, the solution contains not more than 3% w/w of total impurity by weight of Cetrorelix base.

Cetrorelix acetate is thermally labile and hence it is difficult to sterilize by autoclaving, thus requiring sterilisation by aseptic filtration. However, Cetrorelix is unstable in aqueous solution, and tends to aggregate and gel. Therefore, sterilisation by filtration can become troublesome, as there is increase in viscosity due to gel formation. Thus, there is a need to develop a less viscous formulation which is easy to process aseptically, and which can be filtered without formation of gel or aggregates.

According to the present invention, the pharmaceutical composition comprises one or more amino acids as stabilizer. The amino acid stabilizer reduces interactions between peptide molecules, thereby slowing down the rate of aggregation. Also, it helps in solubilization of the peptide. The amino acid stabilizer is selected from the group comprising glycine, methionine, histidine and mixtures thereof. The preferred amino acid stabilizer is glycine. The amount of glycine used may be in the range of about 1 mg/ml to about 10 mg/ml of the composition.

Injectable compositions should be isotonic to human plasma to avoid damage to the tissue. According to the present invention, the pharmaceutical composition also comprises isotonicity agents selected from the group comprising mannitol, sucrose, glycerine, trehalose, sorbitol, glycerol and the like, and mixtures thereof. It may be used in a concentration ranging from about 10 mg/ml to about 40 mg/ml. The osmolality of the compositions of the present invention may be adjusted in the range of about 250 mOsmol to about 500 mOsmol. The preferred isotonicity agent is selected from mannitol and glycerol. The isotonicity agent also helps in maintaining colloidal stability of proteins and peptides through preferential exclusion theory. Hence, it also reduces the aggregation and gel forming propensity of the compositions of the present invention.

In case of Cetrorelix compositions of the present invention, the pH of the composition plays an important role in maintaining the drug in solution. According to the present invention, an acidifying agent, selected from the group comprising acetic acid, citric acid, tartaric acid, succinic acid and mixtures thereof, may be used to provide a composition with a pH in the range of about 3 to about 5.5. In preferred embodiments, the weight ratio of Cetrorelix base to the acidifying agent may be in the range of about 0.01:1 to about 1:1.

According to the present invention, the Cetrorelix base is present in the said composition in an amount ranging from about 0.1 mg to about 0.5 mg/ml of the composition.

According to the present invention, other pharmaceutical excipients which may be added to the said parenteral composition include one or more of chelating agents, reducing agents, antioxidants, buffers, preservatives and the like.

The stable sterile ready-to-use Cetrorelix compositions of the present invention may be packaged in vials, prefilled syringes (PFS) or in cartridges for autoinjectors. The material of construction of the vials, the prefilled syringes and cartridges is such that stability and sterility of the Cetrorelix composition of the present invention is not compromised over its shelf-life. It may be made up of a material selected from glass, plastic or a polymeric material. In preferred embodiments, the material of construction is glass, such as USP Type I siliconized glass or non-pyogenic glass. In other embodiments, the material of construction is non-glass plastic or polymeric material selected from cycloolefin polymer, cycloolefin copolymer, polyolefins, styrene-polyolefin based polymers and block co-polymers, polycarbonates and the like.

The ready-to-use Cetrorelix composition of the present invention, when provided in PFS or cartridges for use with autoinjectors, are suitable for self-administration, or can be administered to the patient by another person with ease. The ready-to-use composition of the present invention may be administered subcutaneously either once daily (0.25 mg dose) or once (3 mg dose) during the early- to mid-follicular phase. The composition is subcutaneously injected in the lower abdominal area, preferably around, but staying at least one inch away from the navel. Accordingly, the present invention provides a ready-to-use composition of Cetrorelix or its pharmaceutically accepted salt, in a single-use PFS or autoinjector device for subcutaneous delivery of the composition to a subject by self-administration.

The term “shelf-life” refers to the amount of time the pharmaceutical composition may be stored without loss of potency and/or performance profile. In some embodiments of the present invention, shelf life refers to the amount of time the pharmaceutical composition may be stored without loss of 2%, 5%, 8% or 10% of the potency and/or performance. The stable compositions provided herein are designed to have shelf-life of at least 12, 24 or 36 months.

The ready-to-use compositions of the present invention, when stored at 2-8° C. exhibit a shelf life of at least about 24 months, as evidenced by accelerated stability studies conducted on the compositions. As can be seen in the examples provided below, the ready-to-use Cetrorelix compositions of the present invention, when stored at 25° C. and 60% relative humidity, have not more than 3% w/w of total impurity at the end of 6 months of storage. Further, the composition contains not more than 1% w/w of L-Ala-Cetrorelix impurity of formula II at the end of 6 months of storage at 25° C. and 60% relative humidity. This data, when extrapolated, indicates a shelf life of at least 24 months. The real time stability data of the composition also indicates a stable composition, wherein less than 0.1% w/w L-Ala Cetrorelix impurity and <0.5% w/w of total impurity was found at the end of 6 months of storage at 2-8° C.

Cetrorelix acetate drug substance is known to contain Endo (3)-D-Pal Cetrorelix (Formula I) as a process impurity. Typically, this impurity is found to increase with time in the finished dosage form or drug product, i.e. the final composition, if the carrier selected is not right. The present invention provides a ready-to-use Cetrorelix composition wherein the carrier or excipients selected are such that the Endo (3)-D-Pal Cetrorelix impurity does not increase with time upon storage, and is found to stay below 0.5% w/w at the end of 6 months of storage at 25° C. and 60% relative humidity.

The present invention also provides process for the preparation of the ready-to-use pharmaceutical compositions of Cetrorelix or its pharmaceutically acceptable salt.

### Definitions

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

All publications, patent applications, patents, and other references mentioned herein are incorporated by reference herein in their entirety for all purposes.

As used herein, “a,” “an,” or “the” can mean one or more than one.

Also, as used herein, “and/or” refers to and encompasses any and all possible combinations of one or more of the associated listed items, as well as the lack of combinations when interpreted in the alternative (“or”).

As used herein, the term “about,” as used herein when referring to a measurable value such as an amount of a compound or agent of this invention, dose, time, temperature, and the like, is meant to encompass variations of +10% of the specified amount.

“Ready-to-use” when used in connection with a Cetrorelix composition refers to a composition that includes Cetrorelix or its pharmaceutically acceptable salt in dissolved or solubilized form, and may be used as such, without further dilution with diluents. The terms “composition” and “solution” may be used interchangeably.

The term “parenteral” as used herein, refers to administration by the subcutaneous or intramuscular route.

As used herein, and unless otherwise specified, the term “stable” refers to physical and chemical stability of the composition.

The present invention is further illustrated by reference to the following examples which is for illustrative purposes only, and does not limit the scope of the invention in any manner.

### Example 1

| Ingredient | Quantity/mL | |
|---|---|---|
| Cetrorelix | 0.25 | mg |
| Acetic acid | 3 | mg |
| Mannitol | 30 | mg |
| Glycine | 7.5 | mg |

| | |
|---|---|
| Water for injection | Qs to 1 mL |

Transfer required quantity of acetic acid for the batch into manufacturing vessel. Accurately weigh and add required quantity of cetrorelix into the manufacturing vessel. Stir the above mixture until clear solution is obtained. Add required quantity of 80% of water for injection required for the batch into above manufacturing vessel and stir the mixture to get uniform solution. Add required quantity of glycine to the solution and stir until clear. Add the required quantity of mannitol and stir. Finally make up the volume to 100% using the remaining amount of water for injection. Filter the solution through 0.2p filter and fill the solution into vials or PFS or cartridge for autoinjector.

### Example 2

| Ingredient | Quantity/mL | |
|---|---|---|
| Cetrorelix | 0.25 | mg |
| Acetic acid | 3 | mg |
| Glycerine | 30 | mg |
| Glycine | 7.5 | mg |

| | |
|---|---|
| Water for injection | Qs to 1 mL |

The composition is obtained by a process similar to that in Example 1 above.

### Example 3

The composition of Example 2 was subjected to stability testing, and the results are provided below.

| | | Condition | | |
|---|---|---|---|---|
| | | | 25 ± 2° C., | 40 ± 2° C., |
| | | 2-8° C. | 60 ± 5% RH | 75 ± 5% RH |
| Parameter | Initial | 2 weeks | 2 Weeks | 2 weeks |
| Description | CCS | CCS | CCS | CCS |
| pH | 3.56 | 3.61 | 3.58 | 3.60 |
| Osmolality (mOsmol) | 511 | NP | NP | NP |
| Assay (%) | 98.78 | 98.39 | 96.80 | 96.28 |
| Related substances by | Impurities % w/w | | | |
| HPLC | | | | |
| unknown @ RRT 0.23 | ND | 0.03 | 0.05 | 0.03 |
| Endo (3) -D-Pal | 0.02 | 0.02 | 0.04 | 0.39 |
| Cetrorelix | | | | |
| unknown @ RRT 0.81 | ND | ND | 0.01 | 0.04 |
| unknown @ RRT 0.89 | 0.07 | 0.05 | 0.04 | 0.01 |
| L-Ala-Cetrorelix | 0.08 | 0.07 | 0.07 | 0.06 |
| unknown @ RRT 1.10 | ND | ND | 0.02 | 0.12 |
| Total Impurities | 0.24 | 0.17 | 0.23 | 0.69 |

RH: Relative humidity;
RRT: Relative Retention Time;
CCS: Clear Colorless solution;
NP: Not Performed;
ND: Not Detected

The structures of the known impurities are as follows—

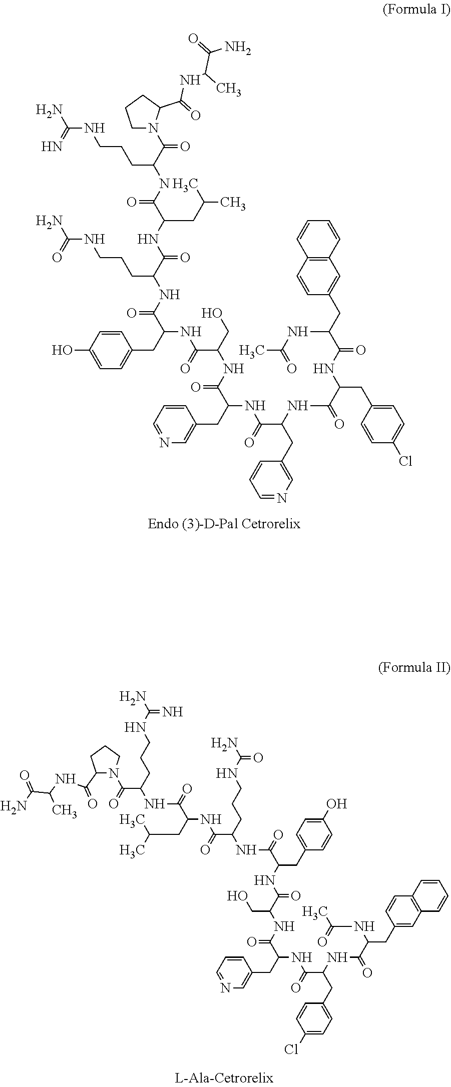 [SMILES: CC(=O)NC(Cc1ccc2ccccc2c1)C(=O)NC(Cc1ccc(Cl)cc1)C(=O)NC(Cc1cccnc1)C(=O)NC(Cc1cccnc1)C(=O)NC(CO)C(=O)NC(Cc1ccc(O)cc1)C(=O)NC(CCCNC(N)=O)C(=O)NC(CC(C)C)C(=O)NC(CCCNC(=N)N)C(=O)N1CCCC1C(=O)NC(C)C(N)=O ; CC(=O)NC(Cc1ccc2ccccc2c1)C(=O)NC(Cc1ccc(Cl)cc1)C(=O)NC(Cc1cccnc1)C(=O)NC(CO)C(=O)NC(Cc1ccc(O)cc1)C(=O)NC(CCCNC(N)=O)C(=O)NC(CC(C)C)C(=O)NC(CCCNC(=N)N)C(=O)N1CCCC1C(=O)NC(C)C(N)=O]

There was no observation of aggregation in the Cetrorelix formulation during manufacturing and subsequent storage.

### EXAMPLE 4

| Ingredient | Qty per mL | |
|---|---|---|
| Cetrorelix acetate as Cetrorelix | 0.25 | mg* |
| Acetic acid | 3.00 | mg |
| Propylene glycol | 10.00 | mg |
| Glycerol | 5.00 | mg |
| Glycine | 2.50 | mg |

| | |
|---|---|
| Water for injection | Q.S. to 1 mL |

Note:
*Cetrorelix acetate is taken as cetrorelix on as is basis.

About 15% of water for injection for the batch was taken in a manufacturing vessel, and accurately weighed acetic acid was added to it. The mixture was stirred until a clear solution was obtained. Accurately weighed quantity of propylene glycol was then added to the vessel and stirred to obtain a clear solution. Cetrorelix acetate was then added to the vessel and stirred to obtain a uniform solution. The required quantity of glycerol was added with stirring to the mixture, followed by addition of glycine. Finally, the volume was made up to 100% using the remaining amount of water for injection. The solution thus obtained was filtered through 0.2p filter and filled into a PFS.

### Example 5

The composition of Example 4 was subjected to stability testing and the results are provided below.

| | | | Storage condition & Timepoint | | | | | | | |
|---|---|---|---|---|---|---|---|---|---|---|
| Name of the | | | | | | | 25 ± 2° C./60% relative | | | |
| test | | | 2-8° C. | | | | humidity | | | |
| parameter | Specification | Initial | 1 M | 2 M | 3 M | 6 M | 1 M | 2 M | 3 M | 6 M |
| Description | Clear | CCS | CCS | CCS | CCS | CCS | CCS | CCS | CCS | CCS |
| | colorless | | | | | | | | | |
| | solution | | | | | | | | | |
| pH | Between | 3.41 | 3.53 | 3.49 | 3.83 | 3.62 | 3.53 | 3.48 | 3.96 | 3.54 |
| | 3.0 and 5.5 | | | | | | | | | |
| Osmolality | Between | 296 | 282 | 287 | 276 | 281 | 279 | 289 | 279 | 282 |
| (mOsmol/kg) | 260 and | | | | | | | | | |
| | 320 | | | | | | | | | |
| Assay (%) | NLT | 103.3 | 104.8 | 105.7 | 105.7 | 107.5 | 105.6 | 103.2 | 101.6 | 106 |
| | 90.0% | | | | | | | | | |
| | and | | | | | | | | | |
| | NMT | | | | | | | | | |
| | 110.0% | | | | | | | | | |
| Related substances by | | % w/w | | | | | | | | |
| HPLC | | | | | | | | | | |
| Unknown | NMT | 0.03 | 0.03 | 0.03 | 0.04 | 0.03 | 0.12 | 0.18 | 0.26 | 0.5 |
| Impurity @ | 1.0% | | | | | | | | | |
| RRT 0.74 | | | | | | | | | | |
| Impurity L- | NMT | 0.07 | 0.08 | 0.08 | 0.08 | 0.07 | 0.09 | 0.07 | 0.08 | 0.08 |
| Ala- | 1.0% | | | | | | | | | |
| Cetrorelix | | | | | | | | | | |
| (RRT 0.91) | | | | | | | | | | |
| Unknown | NMT | ND | 0.01 | 0.02 | 0.09 | 0.11 | 0.12 | 0.19 | 0.35 | 0.89 |
| Impurity @ | 1.0% | | | | | | | | | |
| RRT 1.10 | | | | | | | | | | |
| Maximum | NMT | 0.02 | 0.01 | 0.02 | 0.02 | 0.02 | 0.02 | 0.06 | 0.12 | 0.07 |
| Unspecified | 1.0% | | | | | | | | | |
| Impurity (%) | | | | | | | | | | |
| Total | NMT | 0.15 | 0.19 | 0.17 | 0.25 | 0.26 | 0.46 | 0.66 | 0.89 | 2.02 |
| Impurities | 3.0% | | | | | | | | | |

The results of this accelerated stability testing indicates that the composition has a shelf life of at least 24 months.

### Example 6

| | | |
|---|---|---|
| Ingredient | Qty per mL | % w/v |

| | | | |
|---|---|---|---|
| Cetrorelix acetate as Cetrorelix | 0.25 | mg* | 0.025 |
| Tartaric acid | 1.00 | mg | 0.1 |
| Propylene glycol | 10.00 | mg | 1 |
| Glycerol | 5.00 | mg | 0.5 |
| Glycine | 2.50 | mg | 0.25 |

| | | |
|---|---|---|
| Water for injection | Q.S. to 1 mL | Q.S. to 100% |

The composition was obtained by a process similar to that in Example 4 above.

### Example 7

The composition of Example 6 was subjected to stability testing and the results for the same are provided below.

| | | Storage condition & Timepoint | | | | | | |
|---|---|---|---|---|---|---|---|---|
| | | | | | 25 ± 2° C./60% relative | | | |
| | | 2-8° C. | | | humidity | | | |
| Name of the test parameter | Initial | 1 M | 3 M | 6 M | 1 M | 2 M | 3 M | 6 M |
| Description | CCS | CCS | CCS | CCS | CCS | CCS | CCS | CCS |
| pH | 3.19 | 3.15 | 3.56 | 3.39 | 3.13 | 3.34 | 3.50 | 3.37 |
| Osmolality(mOsmol/kg) | 248 | 239 | 236 | 239 | 239 | 240 | 240 | 235 |
| Assay (%) | 95.8 | 96.9 | 95.4 | 99.5 | 95.4 | 95.2 | 99.4 | 97.9 |
| Related substances by HPLC | % w/w | | | | | | | |
| Unknown Impurity @ RRT 0.74 | ND | 0.03 | 0.04 | 0.04 | 0.10 | 0.16 | 0.26 | 0.43 |
| Impurity L-Ala-Cetrorelix (RRT | 0.04 | 0.08 | 0.07 | 0.08 | 0.08 | 0.05 | 0.06 | 0.08 |
| 0.91) | | | | | | | | |
| Unknown Impurity @ RRT 1.10 | ND | 0.03 | 0.08 | 0.18 | 0.22 | 0.33 | 0.58 | 1.44 |
| Maximum Unspecified Impurity | 0.17 | 0.01 | 0.02 | 0.01 | 0.03 | 0.05 | 0.07 | 0.16 |
| (%) | | | | | | | | |
| Total Impurities | 0.25 | 0.15 | 0.22 | 0.34 | 0.49 | 0.64 | 1.10 | 2.31 |

The results of this accelerated stability testing indicates that the composition has a shelf life of at least 24 months.

1. A stable ready-to-use pharmaceutical composition for parenteral administration comprising
i) from about 0.1 mg/ml to about 0.5 mg/ml Cetrorelix base or a pharmaceutically acceptable salt thereof
ii) glycine as a stabilizing agent,
iii) an acidifying agent in an amount sufficient to adjust the pH of the solution in the range of about 3 to about 5.5
iv) an isotonicity agent and
v) optionally cosolvent
wherein after 6 months of storage at 25° C. and 60% relative humidity, the solution contains not more than 1% w/w of L-Ala-Cetrorelix (Formula II) impurity by weight of Cetrorelix base.

2. The ready-to-use pharmaceutical composition of claim 1, wherein the weight ratio of Cetrorelix base to the acidifying agent is in the range of about 0.01:1 to about 1:1.

3. The ready-to-use pharmaceutical composition of claim 1, wherein the isotonicity agent is present in an amount sufficient to provide osmolality of about 250 mOsmol to about 500 mOsmol.

4. The ready-to-use pharmaceutical composition of claim 3, wherein the isotonicity agent is selected from the group consisting of mannitol, sucrose, glycerine, trehalose, sorbitol, glycerol and mixtures thereof.

5. The ready-to-use pharmaceutical composition of claim 1, wherein the amount of glycine is in the range of about 1 mg/ml to about 10 mg/ml of the composition.

6. The ready-to-use pharmaceutical composition of claim 1, wherein the acidifying agent is selected from the group consisting of acetic acid, citric acid, tartaric acid, succinic acid and mixtures thereof.

7. The ready-to-use pharmaceutical composition of claim 1, wherein the cosolvent is selected from propylene glycol, ethanol, glycerine, polyethylene glycol and mixtures thereof.

8. The ready-to-use pharmaceutical composition of claim 1 wherein the composition is administered subcutaneously.

9. The ready-to-use pharmaceutical composition of claim 1 wherein the composition has a shelf life of at least 24 months when stored at 2-8° C.

10. The ready-to-use pharmaceutical composition of claim 9 wherein the composition comprises 0.25 mg/ml of Cetrorelix acetate, 3 mg/ml of acetic acid, 2.5 mg/ml of glycine, 10 mg/ml of propylene glycol, 5 mg/ml of glycerol and has a pH of about 3 to about 5.5.
